(19) United States
(12) Patent Application Publication
(10) Pub. No.: US 2004/0137511 A1
(43) Pub. Date: Jul. 15, 2004

(54) Method for detecting a mycobacterium tuberculosis specific intein and use in diagnosis of tuberculosis

(76) Inventors: Jean-Michel Masson, Toulouse (FR); Fabrice Lefevre, Nimes (FR); Isabelle Saves, Saint Loup Cammas (FR); Marie-Antoinette Laneelle, Vigoulet-Auzil (FR); Mamadou Daffe, Toulouse (FR)

Correspondence Address: YOUNG & THOMPSON, 745 SOUTH 23RD STREET 2ND FLOOR, ARLINGTON, VA, 22202

(21) Appl. No.: 10/203,927

(22) PCT Filed: Feb. 16, 2001
(86) PCT No.: PCT/FR01/00475

(30) Foreign Application Priority Data
Feb. 17, 2000 (FR) 00/02051

(51) Int. Cl. G01N 33/53

(52) U.S. Cl. 435/7.1

(57) ABSTRACT

The invention concerns a method for detecting and/or quantifying Mycobacterium tuberculosis in a sample, characterised in that it consists in detecting in said sample the presence of an intein inserted at a site whereof the location is Mycobacterium tuberculosis specific using a reagent specific to said location, and optionally in quantifying the detected signal.

The present invention relates to the detection of Mycobacterium tuberculosis for the purpose of diagnosing tuberculosis in a patient. The detection method of the invention is based on searching, in a biological sample from a patient, for an intein specific for Mycobacterium tuberculosis. The invention also relates to a kit for carrying out said method.

Mycobacterium tuberculosis is a strict pathogen of humans also capable of infecting some animal species which live alongside them. It constitutes the agent responsible for human tuberculosis. Infection, mainly aerial, most commonly manifests itself through a pulmonary infection.

Tuberculosis is one of the infections which causes most deaths, and it has been reported that the number of deaths increases each year by 10% (Bloom and Murray, Science (1992), 257, 1055-64). Tuberculosis poses a public health problem since not only have a large number of children in developing countries already been infected, or will be before reaching an adult age, but tuberculosis is also one of the opportunistic infections developed by immunodepressed individuals such as individuals suffering from Aids. Moreover, many strains of Mycobacterium tuberculosis exhibit resistance to various antibiotics (Shankar et al., Lancet (1990), 335, 423-42), which makes treatment all the more difficult.

Conventional techniques for diagnosing Mycobacterium tuberculosis are based on a microscopic examination or on sample culture. Detection by microorganism culture is sensitive but expensive and the time for detection using this method takes a few weeks. Direct staining techniques are, on the other hand rapid, but lack sensitivity and specificity.

Molecular methods, such as PCR amplification or hybridization of probes specific for Mycobacterium tuberculosis, corresponding, for example, to the 16S rRNA subunit, have been widely described for detecting tuberculosis infections.

However, these methods have difficulties, listed in application WO 99/35284. Combined with analysis on polyacrylamide gel, diagnosis by PCR appears to be more effective according to application WO 99/35284. However, this method of diagnosis is illustrated in examples only on samples originating from cell culture.

The aim of the present invention is precisely to provide novel diagnostic means which are rapid, sensitive and specific for infection with Mycobacterium tuberculosis. This aim is achieved according to the invention by virtue of the detection of one or more inteins specific for Mycobacterium tuberculosis.

Inteins are protein introns which are integrated into proteins. The protein splicing of these introns is necessary for survival of the organism to which they belong. Thus, inteins have been described in Mycobacterium tuberculosis in the proteins RecA, Pps1 and DnaB.

U.S. Pat. No. 5,795,731 reports a method for screening for antibiotics or antifungal agents which are capable of inhibiting the protein splicing of the Mycobacterium tuberculosis RecA intein, but that document does not relate to a diagnostic method. In addition, this method for screening for antibiotics involves cloning the RecA intein into a reporter gene beforehand. It is a method which is relatively laborious to carry out and which also requires a cell culture step.

The inventors have now discovered that inteins specific for Mycobacterium tuberculosis exist. They are more particularly inteins liable to be present in several types of mycobacterium, but the location of which is specific for Mycobacterium tuberculosis.

The studies carried out in the context of the present invention have made it possible to identify several inteins, the location of which is specific for Mycobacterium tuberculosis. These are, for example, the inteins located at:

the pps1(b) site of the nucleotide sequence encoding Pps1 (accession No. 2791395),

the dnaB(a) site of the nucleotide sequence encoding DnaB (accession No. 3250719)

the recA(a) site of the nucleotide sequence encoding RecA (accession No. X58485).

Of course, this list is nonlimiting and the invention extends to any intein, the location of which is specific for Mycobacterium tuberculosis.

Thus, the RecA intein of Mycobacterium tuberculosis is located at the recA(a) site whereas the RecA inteins of Mycobacterium leprea, M. chitae, M. fallax, M. flavescens, M. gastri, M. thermoresistibile and M. shimoidei when they are present, are located at the recA(b) site, as shown in table 1 below.

TABLE 1

| | recA | | | | |
|---|---|---|---|---|---|
| | | Intein size | | | Insertion |
| Species | Reference | bp | | (aa) | site |
| M. tuberculosis | X58485 | 1 | 320 | (440) | RecA (a) |
| M. leprea | X73822 | 1 | 095 | (365) | RecA (b) |
| M. chitae | IP 14116001 | +1 | 092 | (364) | RecA (b) |
| M. fallax | CITP 8139 | +1 | 089 | (363) | RecA (b) |
| M. flavescens | ATCC 14474 | +1 | 092 | (364) | RecA (b) |
| M. gastri | HB 4389 | +1 | 104 | (368) | RecA (b) |
| M. thermoresistibile | ATCC 19527 | +1 | 095 | (365) | RecA (b) |
| M. shimoidei | ATCC 27962 | +1 | 092 | (364) | RecA (b) |

The sequence of the recA gene of Mycobacterium tuberculosis with no intein is represented in the attached sequence listing under SEQ ID No. 1, and the sequence of the corresponding protein is represented in the attached sequence listing under SEQ ID No. 2. This sequence comprises, without the intein gene, 1 053 nucleotides. The recA(a) site at which the intein of the recA gene of Mycobacterium tuberculosis is inserted is located between the nucleotides at positions 753 and 754. The sequence of the gene of the intein of the recA gene of Mycobacterium tuberculosis comprises 1 320 nucleotides and is represented in the attached sequence listing under the number SEQ ID No. 3, and the sequence of the corresponding protein is represented under the number SEQ ID No. 4. The sequence of the recA gene of Mycobacterium tuberculosis comprising the intein gene is represented in the attached sequence listing under SEQ ID No. 5, and the sequence of the corresponding protein is represented in the attached sequence listing under SEQ ID No. 6. This sequence comprises 2 373 nucleotides.

The recA(a) intein of Mycobacterium tuberculosis appears to be a multifunctional protein which is not only capable of protein splicing (Davis et al., Cell (1992), 71, 201-10; Kenneth et al., PNAS (1998), 95, 3543-8), but which also has protein ligase activity.

Another intein specific for Mycobacterium tuberculosis is located in the pps1 gene (table 2). The intein of the pps1 gene of Mycobacterium tuberculosis is located at the pps1(b) site, whereas the inteins of the pps1 gene of Mycobacterium leprea and gastri are located, respectively, at pps1(a) and at pps1(c).

TABLE 2

pps1

| | |
|---|---|
| Intein size in | Insertion |

| Species | Reference | Pb | (aa) | site |
|---|---|---|---|---|
| M. tuberculosis | AL123456 | 1 077 | (359) | pps1 (b) |
| M. leprea | U00013 | 1 158 | (386) | pps1 (a) |
| M. gastri | AJ276128 | 1 134 | (378) | pps1 (c) |

The sequence of the pps1 gene of Mycobacterium tuberculosis with no intein is represented in the attached sequence listing under SEQ ID No. 7, and the sequence of the corresponding protein is represented in the attached sequence listing under SEQ ID No. 8. The sequence of the pps1 gene of Mycobacterium tuberculosis comprises, without the intein gene, 1 464 nucleotides. The pps1(b) site at which the specific intein is inserted is located between the nucleotides at positions 756 and 757. The sequence of the intein gene, Mtu Pps1, comprises 1 077 nucleotides and is represented in the attached sequence listing under the number SEQ ID No. 9, and the sequence of the corresponding protein is represented in the attached sequence listing under SEQ ID No. 10. The sequence of the pps1 gene of Mycobacterium tuberculosis comprising the intein gene is represented in the attached sequence listing under SEQ ID No. 11, and the sequence of the corresponding protein is represented in the attached sequence listing under SEQ ID No. 12. The sequence of the gene comprises 2 541 nucleotides.

Like the intein located at recA(a) of Mycobacterium tuberculosis, the intein located at pps1(b) is a multi-functional protein which is not only capable of protein splicing, and therefore of protein ligase activity, but which also has specific endonuclease activity.

The Pps1 intein of Mycobacterium tuberculosis specifically cleaves the DNA sequence overlapping its site of insertion into the pps1 gene (site <40 bp in length), of the nucleotide sequence represented in the attached sequence listing under the number SEQ ID No. 7.

DnaB also has a specific intein located specifically in Mycobacterium tuberculosis. The DnaB intein of Mycobacterium tuberculosis is located at the DnaB(a) site (table 3), whereas the DnaB inteins of Mycobacterium leprea and M. avium are located at DnaB(b).

TABLE 3

| | dnaB | | | |
|---|---|---|---|---|
| | | Intein size in | | Insertion |
| Species | Reference | bp | (aa) | site |
| M. tuberculosis | 3250719 | 1 248 | (416) | DnaB (a) |
| M. leprea | 2949407 | 435 | (145) | DnaB (b) |
| M. avium | TIGR Mav | 1 011 | (337) | DnaB (b) |
| | Contig1044 | | | |

The sequence of the dnaB gene of Mycobacterium tuberculosis with no intein is represented in the attached sequence listing under SEQ ID No. 13, and the sequence of the corresponding protein is represented in the attached sequence listing under SEQ ID No. 14. The sequence of the dnaB gene of Mycobacterium tuberculosis comprises, without the intein gene, 1 377 nucleotides. The dnaB(a) site at which the specific intein is inserted is located between the nucleotides at positions 1 197 and 1 198. The sequence of the Mtu dnaB intein gene comprises 1 248 nucleotides and is represented in the attached sequence listing under the number SEQ ID No. 15, the sequence of the corresponding protein being represented in the attached sequence listing under the number SEQ ID No. 16. The sequence of the dnaB gene of Mycobacterium tuberculosis comprising the intein gene is represented in the attached sequence listing under SEQ ID No. 17. This sequence comprises 2 625 nucleotides. The sequence of the corresponding protein is represented in the attached sequence listing under SEQ ID No. 18.

Like the inteins located in recA(a) and pps1(b) of Mycobacterium tuberculosis, the intein located in DnaB(a) appears to be a multifunctional protein.

The research studies carried out in the context of the present invention have therefore made it possible for the inventors to design a rapid, specific and sensitive method for diagnosing Mycobacterium tuberculosis, based on the specific location of Mycobacterium tuberculosis inteins.

A subject of the invention is therefore a method for detecting and/or quantifying Mycobacterium tuberculosis in a sample, characterized in that the presence of an intein inserted at a site, the location of which is specific for Mycobacterium tuberculosis, is detected in said sample using a reagent which is specific for said location and, optionally, in that the signal detected is quantified.

According to the method of the invention, “intein” is intended to mean both the detection of an intein and the detection of several inteins simultaneously or successively.

The detection of the presence of an intein located at a site which is specific for Mycobacterium tuberculosis may be carried out using any biological technique known to those skilled in the art, which may or may not comprise comparison with controls. Among these techniques, the invention contemplates more particularly techniques of hybridization with labeled probes capable of hybridizing specifically with all or part of the gene encoding an intein if this intein is inserted at a site, the location of which is specific for Mycobacterium tuberculosis. Such probes may be prepared based on the sequences of the sites of the genes at which the sequences encoding the inteins are inserted in Mycobacterium tuberculosis.

They are preferably labeled probes capable of hybridizing specifically with:

part of the sequence encoding the intein, and

a flanking region of the site at which said intein is inserted, and the location of which is specific for Mycobacterium tuberculosis.

By way of example, in the case of the recA intein of Mycobacterium tuberculosis, the presence of the intein of the recA gene at the recA(a) site of Mycobacterium tuberculosis is sought by hybridization with a labeled probe capable of hybridizing specifically with part of the sequence encoding the recA intein and a flanking region of the recA(a) site at which said intein is inserted, and the location of which is specific for Mycobacterium tuberculosis.

The detection may also involve detection by amplification techniques using sequences, in particular primers specific, for the flanking regions of the site of insertion of the intein specific for Mycobacterium tuberculosis. As amplification techniques, mention may, for example, be made of PCR, NASBA, rolling circle, etc.

This embodiment using diagnosis by amplification of inteins specific for Mycobacterium tuberculosis has the advantage that the result can be controlled. In fact, a specific function of the intein, the corresponding gene of which has been amplified, can be tested. This function may, for example, correspond to the endonuclease activity. The specific primers described previously for the detection by amplification techniques will be chosen so as to subsequently allow expression in vivo or in vitro of said intein in order to be able to test its specific activity possibly corresponding to its endonuclease activity.

The detection of an intein, the location of which is specific for Mycobacterium tuberculosis, makes it possible to draw the conclusion of, and diagnose, infection with Mycobacterium tuberculosis.

As there are several specific sites of insertion of inteins into the Mycobacterium tuberculosis genome, the detection of at least two of these specific inteins simultaneously makes it possible to increase the specificity of detection. Advantageously, at least three inteins located specifically in the Mycobacterium tuberculosis genome are detected simultaneously.

A preferred embodiment of the method for diagnosing Mycobacterium tuberculosis according to the present invention consists in expressing, in vitro, the intein inserted at a site, the location of which is specific for Mycobacterium tuberculosis, and then in detecting it and/or quantifying it using a functional test specific for the activity of said intein expressed in vitro.

By way of example, in the case of the Pps1 intein of Mycobacterium tuberculosis, the pps1 intein of Mycobacterium tuberculosis, if it is present at the pps1(b) site, is expressed, in vitro, using the nucleic acids contained in the sample, and then it is detected and/or it is quantified using a functional test specific for the activity of said intein expressed in vitro.

The functional test used in the method of the invention may in particular be based on:

the endonuclease activity of the intein specific for Mycobacterium tuberculosis;

the protein ligase activity of the intein specific for M. tuberculosis;

the protein splicing of the intein specific for M. tuberculosis.

This detection via the function makes it possible to avoid false-positives. In fact, by PCR, it is noted that there is always a risk regarding the nature of the amplicon, which may induce false-positives which are very prejudicial in the context of the diagnosis.

A subject of the invention is therefore most particularly a method for detecting and/or quantifying Mycobacterium tuberculosis in a sample, characterized in that it comprises the following steps:

a) preparing, from said sample, nucleic acid molecules comprising a polynucleotide sequence at least encoding an intein inserted at a site, the location of which is specific for Mycobacterium tuberculosis, and the control elements required for the in vitro transcription and translation of said intein;

b) transcribing and translating, in vitro, the nucleic acid molecules prepared in step (a);

c) detecting and/or measuring a function of the specific intein expressed in step (b).

The sample on which the method of the invention is carried out may be any biological sample liable to contain Mycobacterium tuberculosis. It may, of course, be a crude biological sample such as blood, tissues or a body fluid, for instance sputum, saliva and expectorations. These samples may also correspond to products of any DNA or RNA amplification methods or any nucleic acid products derived from treatment commonly used in the field of biology.

For convenience, the term “mycobacterium” is intended to mean both the microorganism Mycobacterium tuberculosis itself and its genetic information.

In addition, the term “function” is intended to mean any property of the specific intein, such as an enzymatic activity specific to the intein of Mycobacterium tuberculosis. As indicated above, this function may correspond, for example, to the endonuclease activity of the intein or to the protein ligase activity or to its protein splicing capacity.

Advantageously, the detection and/or quantification method according to the invention is carried out in the presence of one or more substances capable of modifying the activity of the intein.

The method of the invention also offers the advantage of being very sensitive. This sensitivity is explained by the multiplying coefficient of steps (b) and (c) corresponding, respectively, to the transcription and to the translation of the gene(s) prepared in step (a), encoding the intein, and then to the detection and/or the measurement of the function corresponding to the protein(s) produced in step (b). In addition, in order to increase the sensitivity, the method of the invention may, after the transcription step, involve a step of amplification of the transcripts by any technique known to those skilled in the art, such as NASBA (nucleic acid sequence-based amplification) or TMA (transcription mediated amplification), before the translation step.

The method of the invention is also rapid and reproducible, since all the reactions are carried out in vitro in a few hours.

The method of the invention makes it possible not only to demonstrate the presence of, and/or to quantify, an intein specific for Mycobacterium tuberculosis, and therefore to specifically diagnose Mycobacterium tuberculosis, but it also makes it possible to characterize said intein. The term “characterization” is, for example, intended to mean definition of the spectrum of inhibition of the specific intein by specific inhibitors or definition of a pH range in which the intein is active. This particular embodiment of the method of the invention makes it possible to define new antibiotics or substances capable of inhibiting functions of the specific intein, and more particularly the protein splicing necessary for survival of the organism to which it belongs.

Given the ease, the low cost and the rapidity of the method of the invention, an infection with M. tuberculosis can be monitored in such a way as to periodically detect and/or quantify a function of the specific intein in an organism which has or has not undergone treatment with an antibacterial agent. Comparison of the results obtained at various times and the interpretation thereof allows the progress of the infection to be monitored over time.

This type of monitoring allows the practitioner to understand the development of an infection with M. tuberculosis. Interpretation of the results then provides a considerable aid to decision-making.

By way of particular inteins, the insertion of which is located at a site specific for Mycobacterium tuberculosis, the invention relates more particularly to the inteins located at:

the pps1(b) site of the nucleotide sequence encoding Pps1 (accession No. 2791395),

the dnaB(a) site of the nucleotide sequence encoding DnaB (accession No. 3250719),

the recA(a) site of the nucleotide sequence encoding RecA (accession No. X58485).

Other advantages and characteristics of the invention will appear in the following description of each of the steps of the preferred method of the invention. For convenience, the term “specific intein” will also denote an intein inserted at a site, the location of which is specific for Mycobacterium tuberculosis.

1) Step (a) of Preparing the Sample.

When the method of the invention consists in detecting Mycobacterium tuberculosis, a nucleic acid molecule encoding a specific intein is prepared in step (a) so as to be able to express it subsequently in vitro.

Most preferentially, when the method of the invention consists in quantifying Mycobacterium tuberculosis, an amount of nucleic acid molecules proportional to the amount of mycobacteria possibly present in the sample is prepared in step (a).

The preparation of the sample in step (a) of the method of the invention consists in placing a nucleic acid sequence encoding at least the intein inserted at a site, the location of which is specific for Mycobacterium tuberculosis, under the control of elements required for the transcription and translation in vitro of said gene.

Thus, in step (a), the regulatory sequences for the polynucleotide encoding the specific intein according to the method of the invention are, for transcription:

an RNA polymerase promoter in 5′

optionally, an RNA polymerase terminator in 3′, and for translation:

a ribosome binding site

a translation initiation codon in phase with the first codon of the intein gene

a translation stop codon followed by some nucleotides, such as, for example, from 5 to 10 nucleotides.

The promoter (in 5′) and the terminator (in 3′), if it is present, of an RNA polymerase are, for example, those of the T7, SP6, Qβ or λ phage RNA polymerase.

An advantageous embodiment of step (a) of the method of the invention consists in preparing the nucleic acid molecule via a reaction to amplify the gene encoding the specific intein, from the nucleic acids of the sample. This may be an amplification by PCR or by PCR-derived techniques, for example nested PCR, or techniques different from PCR, of the NASBA or SDA type, or others. Advantageously, this preparation uses at least two oligonucleotides or at least two primers, two of which are located, respectively, at the edges of the nucleotide sequence encoding the intein inserted at a site, the location of which is specific for Mycobacterium tuberculosis.

This preparation by amplification (for example PCR or NASBA) is carried out using primers which may correspond, for example:

for the sense primer(s), to at least the following elements corresponding to an RNA polymerase promoter, a ribosome binding site, a translation initiation codon in phase with the first codon of the intein gene, and the sequence which hybridizes at least in 5′ of the polynucleotide encoding the specific intein, and

for the antisense primer(s), to at least the following elements comprising the sequence which hybridizes at least in 3′ of the polynucleotide encoding the specific intein, a translation stop codon followed by some nucleotides, such as, for example, 5 to 10, and optionally an RNA polymerase terminator.

The preparation of the nucleic acid molecule of step (a) may be carried out by any other method known to those skilled in the art, such as restriction cleavage making it possible to recover the specific intein of interest, followed by oriented ligation with the regulatory elements required for the transcription and for the translation in vitro indicated above.

When a pair of primers is used in step (a) for preparing nucleic acid molecules, these two primers are capable of hybridizing, respectively, to the edges of the coding sequence for an intein inserted at a site, the location of which is specific for Mycobacterium tuberculosis.

The parts of the primers which hybridize at least to the gene encoding a specific intein are from 7 to 150 nt in length, advantageously from 7 to 50, and preferentially from 10 to 25 nt.

In a particular embodiment, these primers have a segment of at least 7 contiguous bases which are at least 70% complementary to a target sequence of 7 contiguous nucleotides located on both sides of the polynucleotide encoding at least the intein specific for Mycobacterium tuberculosis.

Still in the case when a pair of primers is used in step (a) of preparing nucleic acid molecules, different embodiments of the method of the invention may be implemented. It is possible to combine, possibly in a single tube, at least two detection and/or quantification reactions. One of the detection and/or quantification reaction concerns Mycobacterium tuberculosis. The other “associated” detection and/or quantification reaction concerns an organism or a process which it is useful to detect in parallel to Mycobacterium tuberculosis. This parallel detection also involves the associated function of an organism or of a process, expressed in vitro. This organism may also correspond to a mycobacterium.

2) Step (b) of Transcription and Translation.

The transcription and translation reactions (step b) may be simultaneous, which means that the translation phase is carried out simultaneously with the transcription, or broken down into two different steps of transcription and of translation.

Decoupling allows the use of different translation extracts depending on the origin of the DNA screened. In fact, the transcript translation phase is advantageously carried out with a translation extract of mycobacterial origin or of an origin close to that of the biological sample on which the method of the invention is carried out. The origin of the signals for translation of the transcripts and the cell extract are optimally suited for optimal translation effectiveness. By way of example, mention may be made of the use of a translation extract prepared from nonpathogenic mycobacteria, for translating polynucleotides encoding the intein specific for pathogenic Mycobacterium tuberculosis. These respective extracts are liable to improve the effectiveness of the method. These extracts are chosen for their ability to translate the transcripts.

The method of the invention is notable in that it allows the punctuation of expression of the transcripts and the translation extracts used to be suited to one another. These translation extracts are also characterized in that either they do not contain the property sought, or they contain it but it is not detectable under the test conditions produced to detect the function sought.

A particular embodiment of the method of the invention consists in using, in step (b), a translation extract prepared from a modified bacterial strain. This extract may also correspond to a mixture of several translation extracts prepared from bacterial strains which may or may not be modified. It may, for example, be a translation extract from E. coli overexpressing a chaperon protein A mixed with a translation extract of E. coli overexpressing a chaperon protein B. Any type of mixture can be contemplated as long as it corresponds to the characteristics described above. Similarly, it is possible to use a translation extract to which are added one or more tRNAs specific for one or more codons. The translation extracts thus obtained then make it possible to translate mRNAs comprising these specific codons. The treatment in step (b) with a translation extract may also be carried out with a standard translation extract, whatever the origin of the sample, such as, for example, an extract of E. coli and/or any other cell extract(s) which may or may not be supplemented with advantageous molecules such as those indicated, for example, above (tRNA, chaperon, etc.).

It is also possible to add one or more substances which promote more effective folding or maturation of the proteins expressed, such as, for example, chaperons, detergents, sulfobetaines, membrane extracts, etc., to the translation extract of step (b).

3) Functional Test of Step (c).

In the context of demonstrating the function of the specific intein and of any other protein expressed in step (b), specific substrates may be envisioned by those skilled in the art in order to demonstrate the presence of a function of the specific intein which may correspond, by way of example, to the protein ligase, endonuclease and protein splicing functions. Those skilled in the art may, for example, refer to works such as Methods In Enzymology or Annual Review Of Biochemistry, in which a large number of methods for assaying the activity of proteins and for preparing substrates have been described.

By way of example of detecting the endonuclease activity of the specific Pps1 intein of Mycobacterium tuberculosis, located at the pps1(b) site of the nucleotide sequence, the newly synthesized intein may specifically cleave a polynucleotide, the cleavage of which will cause an emission of fluorescence.

The protein splicing function may be demonstrated by using, for example, during the preparation of the sample by amplification in step (a), the following primers composed:

for the sense primer, of an RNA polymerase promoter, of a ribosome binding site, of part of a reporter gene, such as that encoding microperoxidase (Spee et al., (1996) Eur. J. Biochem 241, 215-220 and Hirayama et al. (1997) Analytical Biochemistry 47, 237-241) and of a sequence which hybridizes in 5′ of the polynucleotide sequence encoding a specific intein, either upstream of the polynucleotide sequence encoding a specific intein or on a sequence upstream of the polynucleotide sequence encoding a specific intein and on the polynucleotide sequence encoding a specific intein, and

for the antisense primer, of the sequence which hybridizes in 3′ of the polynucleotide sequence encoding a specific intein, either downstream of the polynucleotide sequence encoding a specific intein or on the polynucleotide sequence encoding a specific intein and on a sequence downstream of the polynucleotide sequence encoding a specific intein, of the other part of the reporter gene for microperoxidase, of a translation stop codon and, optionally, of an RNA polymerase terminator.

The specific intein may then excise itself from the newly synthesized protein in step (b) so as to release the microperoxidase readily detectable using a functional test in step (c).

The functional test in step (c) may or may not be direct.

The measurement of the function of the specific intein expressed in step (b), if necessary, may be read directly in a fluorimetry reader if measurement of the function uses a substrate corresponding to a fluorophore, or a colorimetry reader if measurement of the function uses a chromophore. However, it is also possible to contemplate measurements by absorbance, by viscosimetry, by mass spectrometry or by any other method related to measurement of the function in step c. It is also possible to contemplate continuous reading of the function, if the latter is suited to this.

4) Quantification of Mycobacterium tuberculosis According to the Invention.

As indicated above, the invention also relates to a method for quantifying the function corresponding to a specific intein based on the nucleic acids present in said sample, characterized in that it comprises:

steps (a) to (c) defined above, step (c) consisting of measuring the function of the specific intein, and then

d) comparing the measurement of the function of the specific intein possibly present in the sample, carried out in step (c), with a standard value or a set of standard values for said function measured on one or more standard samples according to a method of measurement identical or equivalent to that of step (c).

A standard sample for carrying out step (d) above may be any sample containing:

an advantageously known amount of the gene(s) encoding the specific intein which may be transcribed and translated, and which will then be subjected to transcription and translation processing as in step (b), and then the function of said intein will be measured according to a method of measurement identical or equivalent to that of step (c);

an advantageously known amount of the specific intein, which will be measured according to a method of measurement identical or equivalent to that of step (c);

an advantageously known amount of Mycobacterium tuberculosis, which will be measured according to a method of measurement identical or equivalent to that of steps (a), (b) and (c).

The standard sample may originate from a medium identical to or different from that on which steps (a) to (c) of the method of the invention are carried out. It may be the same medium, but taken at a different time.

The detection and/or the quantification may be evaluated in particular relative to a predetermined threshold or relative to a standard curve allowing comparison of the measurements of the function of the specific intein with those of standard samples.

5) Automation and Kit for Carrying Out the Method of the Invention

The steps of the method of the invention may be carried out successively without interruption by the same operator, advantageously on an automated device integrating each one of the steps, or may be carried out discontinuously, optionally by different operators.

The method of the invention may advantageously be automated if the number of samples to be analyzed is high. The nucleic acid samples are then placed on a support, possibly corresponding, for example, to a titration plate or chip, containing several tens to several thousands of positions. These supports are designed to allow:

preparation of the target sequences (step a),

initiation of the reactions for transcription and translation of the specific intein (step b) and revelation of said intein (step c).

Consequently, the invention relates to a device comprising an arrangement of one or more supports, of automated devices and of a reader of said supports, for carrying out the steps of the method described above.

The invention therefore also relates to a kit for carrying out a method for detecting and/or quantifying Mycobacterium tuberculosis described above.

Said kit comprises, in a first embodiment: the means for revealing a function of a specific intein, an RNA polymerase, nucleotide sequences for preparing the nucleic acid molecules (step a) encoding at least the intein, the four nucleotides triphosphate, the mixtures required for said preparation, for the transcription and for the translation, optionally controls and materials for preparing the standards.

In a second embodiment, a kit according to the invention comprises:

optionally, the products and the nucleotide sequences required for step (a) of preparing the polynucleotide sequence encoding at least an intein inserted at a site, the location of which is specific for Mycobacterium tuberculosis,

any support, such as microtitration plate or chip, containing the means for revealing a function of a specific intein, an RNA polymerase, the four nucleotides triphosphate, the transcription and translation mixtures, controls and reagents for preparing standards.

The invention also relates to kits for carrying out a method for detecting the presence of an intein located at a site which is specific for Mycobacterium tuberculosis, using techniques of hybridization with labeled probes capable of hybridizing specifically with all or part of the gene encoding an intein if this intein is inserted at a site, the location of which is specific for Mycobacterium tuberculosis. Said kits contain at least one labeled probe capable of hybridizing specifically with all or part of the gene encoding an intein if this intein is inserted at a site, the location of which is specific for Mycobacterium tuberculosis. Such probes may be prepared based on the sequences of the sites of the genes at which the sequences encoding the inteins are inserted in Mycobacterium tuberculosis. Such labeled probes are advantageously capable of hybridizing specifically with part of the sequence encoding the intein, and a flanking region of the site at which said intein is inserted, and the location of which is specific for Mycobacterium tuberculosis.

The invention also relates to kits for carrying out a method for detecting the presence of an intein located at a site which is specific for Mycobacterium tuberculosis, using amplification techniques. These kits contain at least one pair of primers specific for the flanking regions of the site of insertion of the intein specific for Mycobacterium tuberculosis.

The kits and the supports may be contemplated for detecting and/or quantifying, in addition to M. tuberculosis, one or more other functions associated with one or more other organisms or with one or more processes.

The following description relates to examplary embodiments of the invention concerning (i) the specific detection of Mycobacterium tuberculosis by PCR, and (ii) the specific detection of M. tuberculosis through the function of the intein inserted into the recA(a) site of the gene of the RecA protein.

### EXAMPLE 1

Specific Detection of Mycobacterium tuberculosis by PCR.

The following strains: M. chitae, M. fallax, M. gastri, M. thermoresistible, M. shimoideii were scraped off solid Lowenstein-Jensen medium and resuspended in 1 ml of sterile TE buffer (10 mM Tris-HCl, pH 8, 1 mM EDTA). 1 g of glass beads 0.1 mm in diameter (BioBlock) is added and the mixture is vortexed for 2 min in order to rupture the mycobacteria. Proteinase K (Sigma) is added at a final concentration of 50 μg/ml and the mycobacterial suspensions are incubated for 10 min at 65° C. and then for 10 min at 95° C. The lysates are centrifuged for 10 min at 5 000 g in order to pellet the debris and the glass beads. The supernatant containing the DNA is treated with a freshly prepared solution of phenol/chloroform/isoamyl alcohol (25/28/1) and then with chloroform. These DNA preparations are used directly for the PCR. The M. leprea and M. tuberculosis DNA used was given by S. T. Cole. It is contained in cosmids.

The primers for the PCR amplification reaction were designed in order for them to be able to hybridize on both sides of the recA(a) site in regions conserved for mycobacterial species. These primers correspond to

RecA-3′ (5′AGGATGTCGAACTCGGCCAGCTTGAA 3′) and to

R(a) (5′GCGTCGGTGCGCATGGACGTGCG 3′) and are capable of hybridizing, respectively, to positions 765-791 and 658-681 of the recA gene of Mycobacterium tuberculosis.

The PCR amplification reactions are carried out using:

5 μl of the genomic DNA preparation from M. chitae, M. fallax, M. gastri, M. thermoresistibile and M. shimoidei, prepared as indicated above, and 50 ng of M. tuberculosis and M. leprea cosmid preparation, as DNA matrix,

10 pmol of each oligo,

0.2 mM of dNTP,

5 μl of 10× reaction buffer (100 mM Tris-HCl, pH 8.3, 15 mM MgCl₂, 500 mM KCl),

1 u Taq DNA polymerase, in a final volume of 50 μl.

The amplification cycle is as follows:

10 min at 92° C.

30 cycles (1 min at 92° C.+1 min at 45° C.+1 min 30 at 72° C.)

5 min at 72° C.

The amplification products were loaded onto 2% agarose gel.

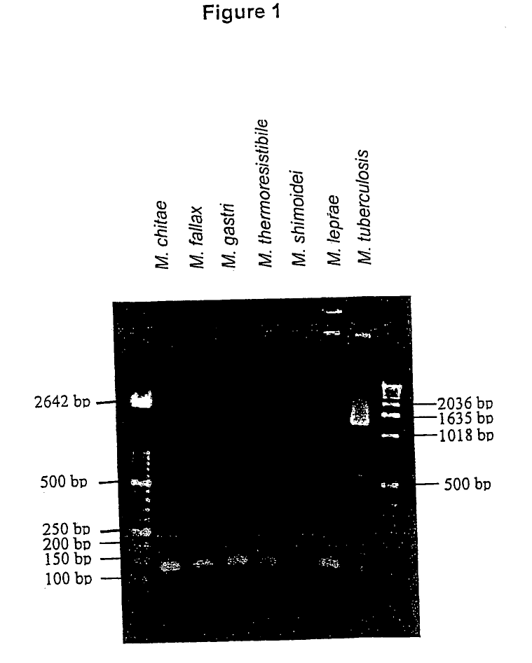

After migration and staining with ETB, the results given in FIG. 1 were observed.

A 133 bp amplification product is observed when the matrix corresponds to M. chitae, M. fallax, M. gastri, M. thermoresistibile, M. shimoidei and M. leprea, whereas a 1 453 bp amplification product is observed if the matrix corresponds to M. tuberculosis. Only the matrix corresponding to M. tuberculosis makes it possible to amplify a 1 453 bp fragment corresponding to 133 bp+the size of the intein inserted at the recA(a) site, namely 1 320 bp.

The presence of M. tuberculosis in a sample can therefore be demonstrated using primers specific for the recA(a) site of insertion of the RecA intein of this mycobacterium.

### EXAMPLE 2

Specific Detection of M. tuberculosis Through the Function of the Intein Inserted into the pps1(b) Site of the Gene of the Pps1 Protein.

A particular embodiment of the invention consists in detecting Mycobacterium tuberculosis via the intein inserted into the pps1(b) site of the gene of the Pps1 protein. For this, the intein gene is amplified by PCR on the genomic DNA of M. tuberculosis using a set of primers making it possible to place this gene under the control of the T7 phage RNA polymerase transcriptional promoter, of a ribosome binding site and of an ATG. The reverse primer comprises one or two STOP codons. In parallel, a negative control is carried out by performing the same PCR on the genomic DNA of a microorganism other than M. tuberculosis.

10 μl of each of these PCRs are then added separately to a 50 μl transcription mixture as described by Pokrovskaya et al. (1994, Analytical Biochemistry, 220, 420-423) at 37° C. for 2 to 3 hours. 10 μl of each of these transcription reactions are then added separately to a translation mixture of final volume 100 μl as described by Zubay (1973, Ann. Revendication. Genet. 7, 267-287) (this translation mixture making it possible to ultimately detect the activity of the intein, and therefore comprising no or very little similar or interfering activity such as endonucleases or exonucleases, etc.), and the reaction is incubated at 37° C. for 2 hours.

1 to 10 μl of each of these translation reactions are incubated independently in a buffered solution allowing restriction digestion (from 50 to 100 μl final volume), in the presence of a linearized plasmid comprising the intein cleavage site. After 20 min to 1 hour of incubation, a fraction of this restriction reaction (from which the proteins have optionally been removed by extraction with phenol-chloroform) is loaded onto a 1% agarose TAE gel. The in vitro translation expressing the negative control shows no modification of the linearized plasmid, whereas that expressing the intein of M. tuberculosis shows cleavage of this plasmid into two bands.

### EXAMPLE 3

Detection of an Intein Specific for Mycobacterium tuberculosis Using Detection of the Endonuclease Activity of Pps1 Intein, the Corresponding Gene of which is Inserted Specifically at the pps1(b) Site.

a) PCR Amplification of the Pps1 Intein of Mycobacterium tuberculosis.

The intein gene is amplified by PCR from genomic DNA with the following primers:

MtuPps1-ATG: 5′ atgtgcctgcccgccggc 3′ and

MtuPps1-3′SS: 5′ gttgtgcacggcgaacccgt 3′.

Genomic DNA is incubated in the presence of 10 pmol of each primer and of Taq DNA polymerase in 10 mM Tris-HCl buffer, pH 8.3, 1.5 mM MgCl₂, 50 mM KCl, 0.2 mM dNTP. The amplification cycle is: 10 min at 92° C.+29 cycles (1 min at 92° C.+1 min at 55° C.+1.5 min at 72° C.)+5 min at 72° C. to finish.

It is possible to observe a 1 077 bp fragment corresponding to the specific amplification of the Pps1 intein inserted at the pps1(b) site.

b) Cloning of the Intein Gene.

The 1 077 bp fragment corresponding to the intein gene is purified on 1% TBE gel using the Qiaquick gel extraction kit (Qiagen), and then inserted into the expression vector pCR-T7-CT-topo according to the manufacturer's recommendations (Invitrogen).

c) Expression and Extraction of the Intein.

BL21-DE3-pLysS bacteria are transformed with a few ng of the expression plasmid and selected on LB medium containing ampicillin and chloramphenicol. A clone is taken up in the same medium and cultured until the exponential growth phase at 37° C., before induction of intein expression with IPTG (1 mM). This induction at 37° C. lasts 2 h 30, the cells are then centrifuged and the proteins extracted in 20 mM sodium phosphate buffer with 6 freezing-thawing cycles.

The protein extract is then recovered by centrifugation of the cell debris.

d) Construction of the DNA Substrate for the Endonuclease Activity of the Pps1 Intein of Mycobacterium tuberculosis.

1 nmol of each oligonucleotide SiteMtuP-Hind

(5′
agcttttgtagatcggtgcggtgcagccctctacgtagtgcacgtt 3′)
and SiteMtuP-Xba
(5′
ctagaacgtgcactacgtagagggctgcaccgcaccgatctacaaa 3′)

are hybridized in 10 mM Tris-HCl buffer, pH 7.5, 100 mM NaCl by incubating them for 5 min at 95° C. and allowing them to return to ambient temperature slowly. This DNA is then ligated to the DNA ligase in the vector pUC19 digested beforehand with the HindIII and XbaI enzymes. The resulting plasmid is a substrate for the endonuclease activity of the intein.

e) Test for the Endonuclease Activity of the Pps1 Intein of Mycobacterium tuberculosis.

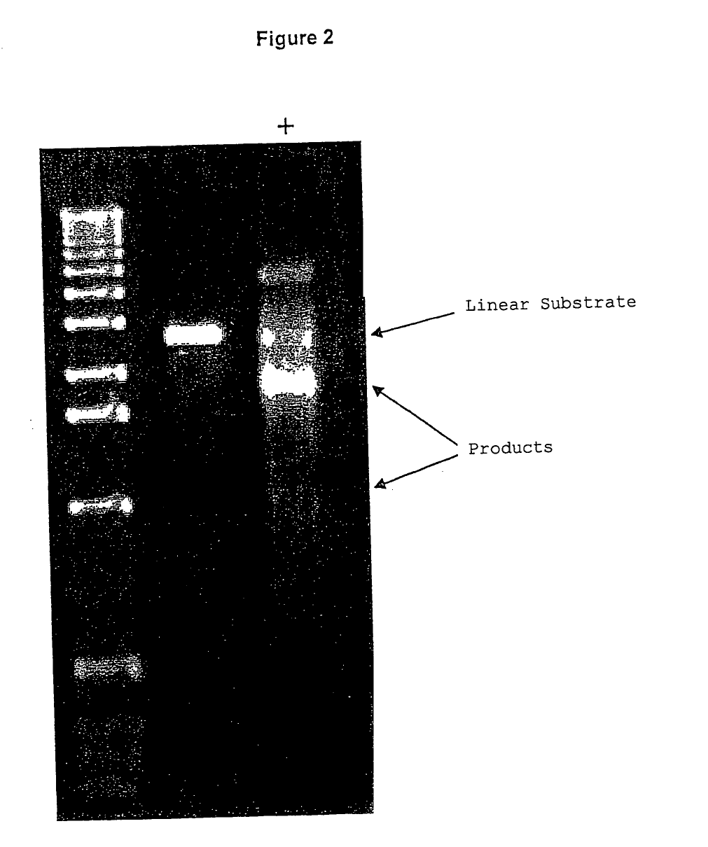

The substrate plasmid is linearized with the ScaI enzyme and diluted to the concentration of 100 ng/μl. For this test, two independent substrate preparations are used (FIG. 2).

100 ng of linear substrate are incubated for 1 h at 37° C. in the presence of intein extract prediluted to 1/20, in 10 mM Tris-HCl buffer, pH 8, 10 mM MgCl₂ and 25 mM KCl. As shown in lane (+) of FIG. 2, the substrate plasmid (2 730 bp) is cleaved into two products (940 and 1 790 bp).

100 ng of linear substrate are incubated for 1 h at 37° C. in the presence of extract of E. coli not expressing the intein, prediluted to 1/20, in 10 mM Tris-HCl buffer, pH 8, 10 mM MgCl₂ and 25 mM KCl (lane (−) of FIG. 2).

It appears that the endonuclease activity of the intein isolated is capable of specifically hydrolyzing the substrate prepared in step c). Mycobacterium tuberculosis is therefore detected in the sample tested.

### EXAMPLE 4

PCR Diagnosis of Mycobacterium tuberculosis through the Simultaneous Detection of Two Specific Inteins: the pps1 Intein and the recA Intein.

The pps1 intein gene is amplified by PCR from genomic DNA of M. gadium and M. tuberculosis with the following primers:

pps1-3′:5′ gtcgttgttcgaccagttctggatggt 3′
pps1-5′:5′ catccgcaacacctacgaccgg 3′.

Genomic DNA of M. gadium and M. tuberculosis is incubated in the presence of 10 pmol of each primer and of one unit of Taq DNA polymerase in 10 mM Tris-HCl buffer, pH 8.3, 1.5 mM MgCl₂, 50 mM KCl, 0.2 mM dNTP. The amplification cycle is: 10 min at 92° C.+29 cycles (1 min at 92° C.+1 min at 50° C.+1.5 min at 72° C.)+5 min at 72° C. to finish.

The recA intein gene is amplified by PCR from genomic DNA of M. leprea and M. tuberculosis with the following primers:

| | |
|---|---|
| RecA-3′ | (5′-aggatgtcgaactcggccagcttgaa 3′) and |
| R (a) | (5′gcgtcggtgcgcatggacgtgcg 3′). |

50 ng of M. tuberculosis and M. leprea cosmid preparation, as DNA matrix, are incubated in the presence of 10 pmol of each primer and of 1 unit of Taq DNA polymerase in 10 mM Tris-HCl buffer, pH 8.3, 1.5 mM MgCl₂, 50 mM KCl, 0.2 mM dNTP. The amplification cycle is: 10 min at 92° C.+30 cycles (1 min at 92° C.+1 min at 45° C.+1.5 min at 72° C.)+5 min at 72° C. to finish.

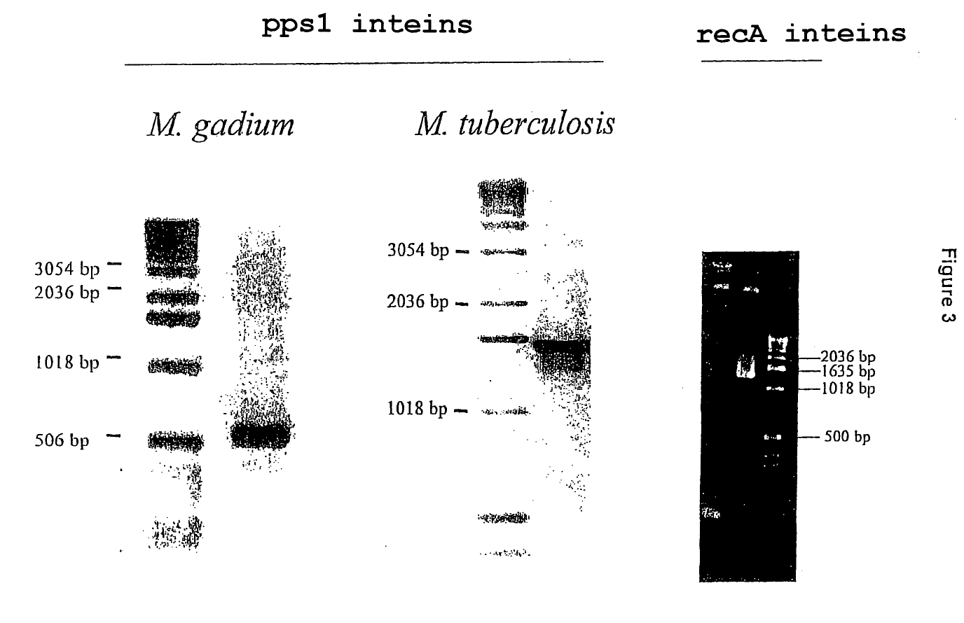

The amplification products were loaded onto agarose gel. After migration and staining with ETB, the results given in FIG. 3 were observed.

Only the matrix corresponding to M. tuberculosis makes it possible to amplify a 1 592 bp fragment corresponding to the specific amplification of the Pps1 intein inserted at the pps1(b) site and a 1 453 bp fragment corresponding to 133 bp+the size of the recA intein inserted at the recA(a) site, namely 1 320 bp.

Detection of the pps1 and recA inteins of Mycobacterium tuberculosis makes it possible to increase the specificity of detection.

1. A method for detecting and/or quantifying Mycobacterium tuberculosis in a sample, characterized in that the presence of an intein inserted at a site, the location of which is specific for Mycobacterium tuberculosis, is detected in said sample using a reagent which is specific for said location and, optionally, in that the signal detected is quantified.

2. The method for detecting and/or quantifying Mycobacterium tuberculosis in a sample as claimed in claim 1, characterized in that the presence of an intein inserted at a site chosen from:
the pps1(b) site of the nucleotide sequence encoding Pps1,
the dnaB(a) site of the nucleotide sequence encoding DnaB,
the recA(a) site of the nucleotide sequence encoding RecA,
is detected in said sample.

3. The method for detecting and/or quantifying Mycobacterium tuberculosis in a sample as claimed in either of claims 1 and 2, characterized in that the presence of at least two or of at least three inteins inserted at a site, the location of which is specific for Mycobacterium tuberculosis, is detected in said sample.

4. The method for detecting and/or quantifying Mycobacterium tuberculosis in a sample as claimed in any one of the preceding claims, characterized in that the presence of an intein inserted at a site, the location of which is specific for Mycobacterium tuberculosis, is detected by hybridization with a labeled probe capable of hybridizing specifically with all or part of the gene encoding an intein if this intein is inserted at a site, the location of which is specific for Mycobacterium tuberculosis.

5. The method for detecting and/or quantifying Mycobacterium tuberculosis in a sample as claimed in any one of the preceding claims, characterized in that the presence of an intein located at a site which is specific for Mycobacterium tuberculosis is detected by amplification using primers specific for the flanking regions of the site for insertion of the intein specific for Mycobacterium tuberculosis.

6. The method for detecting and/or quantifying Mycobacterium tuberculosis as claimed in any one of the preceding claims, characterized in that the intein inserted at a site, the location of which is specific for Mycobacterium tuberculosis, is expressed in vitro, and then in that it is detected and/or quantified using a functional test specific for the activity of said intein expressed in vitro.

7. The method for detecting and/or quantifying Mycobacterium tuberculosis in a sample as claimed in claim 6, characterized in that it comprises the following steps:
a) preparing, from said sample, nucleic acid molecules comprising a polynucleotide sequence at least encoding an intein inserted at a site, the location of which is specific for Mycobacterium tuberculosis, and the control elements required for the in vitro transcription and translation of said intein;
b) transcribing and translating, in vitro, the nucleic acid molecules prepared in step (a);
c) detecting and/or measuring a function of the specific intein expressed in step (b).

8. The method for detecting and/or quantifying Mycobacterium tuberculosis in a sample as claimed in either of claims 6 or 7, characterized in that the function detected and/or measured is based on:
the endonuclease activity of the intein specific for Mycobacterium tuberculosis;
the protein ligase activity of the intein specific for M. tuberculosis;
the protein splicing of the intein specific for M. tuberculosis.

9. The method for detecting and/or quantifying Mycobacterium tuberculosis in a sample as claimed in any one of claims 6 to 8, characterized in that said detection and/or quantification is carried out in the presence of one or more substances capable of modifying the activity of said intein.

10. The method for detecting and/or quantifying Mycobacterium tuberculosis in a sample as claimed in one of claims 7 to 9, characterized in that, in step (a), said regulatory elements are, for transcription:
an RNA polymerase promoter in 5′,
optionally, an RNA polymerase terminator in 3′, and for translation:
a ribosome binding site
a translation initiation codon in phase with the first codon of the intein gene
a translation stop codon followed by some nucleotides, such as, for example, from 5 to 10 nucleotides.

11. A kit for carrying out a method as claimed in any one of claims 7 to 10, characterized in that it comprises: the means for revealing a function of the intein, an RNA polymerase, nucleotide sequences for preparing the nucleic acid molecules of step (a) encoding at least the intein, the four nucleotides triphosphate, the mixtures required for said preparation, for the transcription and for the translation, optionally, controls and standards.

12. A kit for carrying out a method as claimed in any one of claims 7 to 10, characterized in that it comprises:
optionally, the products and the nucleotide sequences required for step (a) of preparing the polynucleotide sequence encoding at least an intein inserted at a site, the location of which is specific for Mycobacterium tuberculosis,
any support, such as microtitration plate or chip, containing the means for revealing a function of a specific intein, an RNA polymerase, the four nucleotides triphosphate, the transcription and translation mixtures, controls and reagents for preparing standards.

13. A kit for carrying out a method as claimed in claim 4, characterized in that it comprises at least one labeled probe capable of hybridizing specifically with all or part of the gene encoding an intein if this intein is inserted at a site, the location of which is specific for Mycobacterium tuberculosis.

14. A kit for carrying out a method as claimed in claim 5, characterized in that it comprises at least one pair of primers specific for the flanking regions of the site of insertion of the intein specific for Mycobacterium tuberculosis.
